(19) United States
(12) Patent Application Publication
(10) Pub. No.: US 2016/0152532 A1
(43) Pub. Date: Jun. 2, 2016

(54) METHOD FOR OXIDATIVELY DEHYDROGENATING N-BUTENES INTO 1,3-BUTADIENE

(71) Applicant: BASF SE, Ludwigshafen (DE)

(72) Inventors: Philipp GRÜNE, Mannheim (DE); Gauthier Luc Maurice AVERLANT, Frankfurt (DE); Ragavendra Prasad BALEGEDDE RAMACHANDRAN, Limburgerhof (DE); Jan Pablo JOSCH, Neustadt (DE)

(21) Appl. No.: 14/905,569

(22) PCT Filed: Jul. 17, 2014
(86) PCT No.: PCT/EP2014/065373

§ 371 Date: Jan. 15, 2016

(30) Foreign Application Priority Data
Jul. 18, 2013 (EP) 13177103.2

(51) Int. Cl. C07C 5/48; C07C 7/11

(52) U.S. Cl. CPC C07C 5/48; C07C 7/11; C07C 2523/887

(57) ABSTRACT

A process for oxidative dehydrogenation of n-butenes to 1,3-butadiene in a fixed-bed reactor (R), which comprises at least two production steps (i) and at least one regeneration step (ii), and in which
in a production step (i), a starting gas mixture (1) comprising the n-butenes is mixed with an oxygen-comprising gas (2) and brought into contact with a heterogeneous, particulate multimetal oxide catalyst comprising molybdenum and at least one further metal as active composition in the fixed-bed reactor (R) and
in a regeneration step (ii), the heterogeneous, particulate multimetal oxide catalyst comprising molybdenum and at least one further metal as active composition is regenerated by passing an oxygen-comprising regeneration gas mixture over it and burning off the carbonaceous material deposited on the multimetal oxide catalyst,
   where
   a regeneration step (ii) is carried out between two production steps (i) and where
a product gas stream (6) which
comprises 1,3-butadiene and additionally unreacted n-butenes, oxygen, water and further secondary components, in particular carbon monoxide, carbon dioxide, inert gases, in particular nitrogen, high-boiling hydrocarbons, i.e. hydrocarbons having a boiling point of 95° C. or above at a pressure of one atmosphere, optionally hydrogen and optionally oxygenates is obtained in the production step (i) in the fixed-bed reactor (R) and is fed as such or after one or more intermediate steps as stream (11)
to an absorption column (K) in which an absorption is carried out at a pressure in the range from 3.5 to 20 bar by means of a high-boiling absorption medium (13) which becomes loaded with the C4-hydrocarbons from the product gas stream (6) or the stream (11) and is taken off as loaded solvent stream (14) from the bottom of the absorption column (K) to give an overhead stream (12) comprising
   oxygen, low-boiling hydrocarbons, i.e. hydrocarbons having a boiling point of less than 95° C. at a pressure of one atmosphere, residues of C4-hydrocarbons, residues of high-boiling hydrocarbons, i.e. hydrocarbons having a boiling point of 95° C. or above at a pressure of one atmosphere, optionally inert gases, in particular nitrogen, optionally carbon oxides and optionally water vapor, and is partly or completely
   recycled as recycle stream to the fixed-bed reactor (R),
   wherein
   at the end of each production step (i), the introduction of the oxygen-comprising gas (2) into the reactor (R) is throttled back or shut off and the production step (i) is continued until the oxygen concentration in the overhead stream (12) has decreased to 5% by volume, based on the total volume of the overhead stream (12), whereupon
the introduction of the gas stream (1) comprising the n-butenes
and also the introduction of the oxygen-comprising gas (2) is shut off, if this has not already been done at the end of the production step (i),
   at which point the production step (i) is complete and the regeneration step (ii) is started by the overhead stream (12) from the absorption column (K) functioning as oxygen-comprising regeneration gas mixture or substream of the oxygen-comprising regeneration gas mixture, is proposed.

### CROSS-REFERENCE TO RELATED APPLICATIONS

This application is a national stage application (under 35 U.S.C. §371) of PCT/EP2014/065373, filed Jul. 17, 2014, which claims benefit of European Application No. 13177103.2, filed Jul. 18, 2013, both of which are incorporated herein by reference in their entirety.

The invention relates to a process for the oxidative dehydrogenation of n-butenes to 1,3-butadiene.

1,3-Butadiene is an important basic chemical and is used, for example, for the preparation of synthetic rubbers (butadiene homopolymers, styrene-butadiene rubber or nitrile rubber) or for the preparation of thermoplastic terpolymers (acrylonitrile-butadiene-styrene copolymers). 1,3-Butadiene is also converted into sulfolane, chloroprene and 1,4-hexamethylenediamine (via 1,4-dichlorobutene and adiponitrile). Furthermore, 1,3-butadiene can be dimerized to produce vinylcyclohexene which can be dehydrogenated to form styrene.

1,3-Butadiene can be prepared by thermal cracking (steam cracking) saturated hydrocarbons, with naphtha usually being used as raw material. The steam cracking of naphtha gives a hydrocarbon mixture of methane, ethane, ethene, acetylene, propane, propene, propyne, allene, butanes, n-butenes, 1,3-butadiene, butynes, methylallene, C₅-hydrocarbons and higher hydrocarbons.

1,3-Butadiene can also be obtained by oxidative dehydrogenation of n-butenes (1-butene and/or 2-butene). Any mixture comprising n-butenes can be used as starting gas mixture for the oxidative dehydrogenation of n-butenes to 1,3-butadiene. For example, it is possible to use a fraction which comprises n-butenes (1-butene and/or 2-butene) as main constituent and has been obtained from the C₄ fraction from a naphtha cracker by removal of 1,3-butadiene and isobutene. Furthermore, gas mixtures which comprise 1-butene, cis-2-butene, trans-2-butene or mixtures thereof and have been obtained by dimerization of ethylene can also be used as starting gas. In addition, gas mixtures which comprise n-butenes and have been obtained by fluid catalytic cracking (FCC) can be used as starting gas.

Gas mixtures which comprise n-butenes and are used as starting gas in the oxidative dehydrogenation of n-butenes to 1,3-butadiene can also be prepared by nonoxidative dehydrogenation of gas mixtures comprising n-butane.

WO 2009/124945 discloses a coated catalyst for the oxidative dehydrogenation of 1-butene and/or 2-butene to 1,3-butadiene, which can be obtained from a catalyst precursor comprising

(a) a support body and

(b) a shell comprising (i) a catalytically active multimetal oxide which comprises molybdenum and at least one further metal and has the general formula

Mo₁₂BiₐCr_{b}X¹_{c}Fe_{d}X²ₑX³_{f}O_{y}

where
X¹═Co and/or Ni,
X²═Si and/or Al,
X³═Li, Na, K, Cs and/or Rb,
0.2≦a≦1,
0≦b≦2,
2≦c≦10,
0.5≦d≦10,
0.5≦e≦10,
0≦f≦0.5 and
y=a number which is determined by the valence and abundance of the elements other than oxygen in order to achieve charge neutrality,
   and (ii) at least one pore former.

WO 2010/137595 discloses a multimetal oxide catalyst for the oxidative dehydrogenation of alkenes to dienes, which comprises at least molybdenum, bismuth and cobalt and has the general formula

MoₐBi_{b}Co_{c}Ni_{d}FeₑX_{f}Y_{g}ZₕSiᵢOⱼ

In this formula, X is at least one element selected from the group consisting of magnesium (Mg), calcium (Ca), zinc (Zn), cerium (Ce) and samarium (Sm). Y is at least one element selected from the group consisting of sodium (Na), potassium (K), rubidium (Rb), cesium (Cs) and thallium (TI). Z is at least one element selected from the group consisting of boron (B), phosphorus (P), arsenic (As) and tungsten (W). a-j are the atom fractions of the respective element, where a=12, b=0.5-7, c=0-10, d=0-10, (where c+d=1-10), e=0.05-3, f=0-2, g=0.04-2, h=0-3 and i=5-48. In the examples, a catalyst having the composition Mo₁₂Bi₅Co_{2.5}Ni_{2.5}Fe_{0.4}Na_{0.35}B_{0.2}K_{0.08}Si₂₄ in the form of pellets having a diameter of 5 mm and a height of 4 mm is used in the oxidative dehydrogenation of n-butenes to 1,3-butadiene.

In the oxidative dehydrogenation of n-butenes to 1,3-butadiene, precursors of carboneous material, for example styrene, anthraquinone and fluorenone, which can ultimately lead to carbonization and deactivation of the multimetal oxide catalyst can be formed. The formation of carbon-comprising deposits can increase the pressure drop over the catalyst bed. It is possible to burn off the carbon deposited on the multimetal oxide catalyst at regular intervals by means of an oxygen-comprising gas for the purpose of regeneration, in order to restore the activity of the catalyst.

JP 60-058928 describes the regeneration of a multimetal oxide catalyst for the oxidative dehydrogenation of n-butenes to 1,3-butadiene, which comprises at least molybdenum, bismuth, iron, cobalt and antimony, by means of an oxygen-comprising regeneration gas mixture at a temperature of from 300 to 700° C., preferably from 350 to 650° C., and an oxygen concentration of from 0.1 to 5% by volume. Air which is diluted with suitable inert gases such as nitrogen, steam or carbon dioxide is introduced as oxygen-comprising gas mixture.

WO 2005/047226 describes the regeneration of a multimetal oxide catalyst for the partial oxidation of acrolein to acrylic acid, which comprises at least molybdenum and vanadium, by passing an oxygen-comprising gas mixture over the catalyst at a temperature of from 200 to 450° C. Lean air comprising from 3 to 10% by volume of oxygen is preferably used as oxygen-comprising regeneration gas mixture. Apart from oxygen and nitrogen, the gas mixture can comprise steam.

Neither of the two documents gives any indication as to how such a preferred regeneration gas mixture can be provided inexpensively and reliably. In a shell-and-tube reactor, it is not readily possible to predict the amount and local distribution of the carboneous material. In an unfavorable case, a reaction tube is so strongly carbonized that virtually no flow occurs through it. Heat removal is then greatly hindered and is largely determined by the oxygen content in the regeneration gas mixture. If the oxygen content is high, this can lead to large local temperature increases in the reactor tube, which can lead to destruction of the catalyst or even of the reaction tube and the entire reactor.

Furthermore, the above documents give no indication as to how switching over from a production step to a regeneration step and vice versa should be effected without there being a risk of generating an explosive atmosphere in the reactor or in other regions of the plant.

An inexpensive and readily available oxygen-comprising regeneration gas mixture is air. However, the oxygen content of air is very high at about 20.95% by volume and the possible local temperature increase is therefore very high. An oxygen-enriched regeneration gas mixture can be obtained by diluting air with inert gases such as water vapor, N₂ or Ar. However, a high water vapor content in the regeneration gas mixture can damage the catalyst. Pure inert gases such as N₂ or Ar for dilution are disadvantageous from a financial point of view. Furthermore, large and expensive containers are necessary to keep a sufficient amount of inert gas in stock.

A likewise advantageous and oxygen-depleted gas mixture is the recycle gas obtained by separating off the incondensable or low-boiling gas constituents of the product gas from the oxydehydrogenation. However, in a typical operating state, the recycle gas comprises 5-9% by volume of oxygen, so that the possible local temperature increase is still very high. The direct use of the recycle gas as oxygen-comprising regeneration gas mixture is therefore not advantageous from a safety point of view.

It was an object of the invention to make available a process for the oxidative dehydrogenation of n-butene to 1,3-butadiene, in which the provision of a suitable regeneration gas for the multimetal oxide catalyst is very safe, reliable and inexpensive.

The object is achieved by a process for oxidative dehydrogenation of n-butenes to 1,3-butadiene in a fixed-bed reactor, which comprises at least two production steps and at least one regeneration step and in which
in a production step, a starting gas mixture comprising the n-butenes is mixed with an oxygen-comprising gas and brought into contact with a heterogeneous, particulate multimetal oxide catalyst comprising molybdenum and at least one further metal as active composition in the fixed-bed reactor and
in a regeneration step, the heterogeneous, particulate multimetal oxide catalyst comprising molybdenum and at least one further metal as active composition is regenerated by passing an oxygen-comprising regeneration gas mixture over it and burning off the carbonaceous material deposited on the multimetal oxide catalyst, where
   a regeneration step is carried out between two production steps and
a product gas stream which comprises 1,3-butadiene and additionally unreacted n-butenes, oxygen, water and further secondary components, in particular carbon monoxide, carbon dioxide, inert gases, in particular nitrogen, high-boiling hydrocarbons, i.e. hydrocarbons having a boiling point of 95° C. or above at one atmosphere, optionally hydrogen and optionally oxygenates is obtained in the production step in the fixed-bed reactor and is fed as such or after one or more intermediate steps as stream
to an absorption column in which an absorption is carried out at a pressure in the range from 3.5 to 20 bar by means of a high-boiling absorption medium which becomes loaded with the C₄-hydrocarbons from the product gas stream or the stream and is taken off as loaded solvent stream from the bottom of the absorption column to give an overhead stream comprising
oxygen, low-boiling hydrocarbons, i.e. hydrocarbons having a boiling point of less than 95° C. at one atmosphere, residues of C₄-hydrocarbons, residues of high-boiling hydrocarbons, i.e. hydrocarbons having a boiling point of 95° C. or above at one atmosphere, optionally inert gases, in particular nitrogen, optionally carbon oxides and optionally water vapor, and is partly or completely recycled as recycle stream to the fixed-bed reactor,
wherein
at the end of each production step, the introduction of the oxygen-comprising gas into the reactor is throttled back or shut off and the production step is continued until the oxygen concentration in the overhead stream has decreased to 5% by volume, based on the total volume of the overhead stream, whereupon
the introduction of the gas stream comprising the n-butenes
and also the introduction of the oxygen-comprising gas is shut off, if this has not already been done at the end of the production step, at which point the production step is complete and the regeneration step is started by the overhead stream from the absorption column functioning as oxygen-comprising regeneration gas mixture or substream of the oxygen-comprising regeneration gas mixture.

### A BRIEF DESCRIPTION OF THE FIGURE

FIG. 1 shows a preferred embodiment of a plant for carrying out the process of the invention.

The above process is preferably carried out continuously. The reactor is operated in the production step until, for example, deactivation of the catalyst has reached a particular, prescribed value and, for example, the conversion at constant reaction temperature has dropped by 20%, preferably 10%, preferably 5% and particularly preferably 2%. Furthermore, the reactor can be operated in the production step until the pressure drop over the reactor has increased by a particular, prescribed value, for example by 1000 bar, preferably 500 mbar, preferably 100 mbar and preferably 20 mbar, or until a particular, prescribed duration of the production step has elapsed, for example 2000 h, preferably 1000 h, preferably 500 h and particularly preferably 340 h.

According to the invention, the introduction of the oxygen-comprising gas into the fixed-bed reactor is throttled back at the end of a production step and the oxygen content of the overhead stream from the absorption column thereby functions as oxygen-comprising regeneration gas mixture or substream of the oxygen-comprising regeneration gas mixture and is reduced to a desired concentration of not more than 5% by volume.

Preference is given to the introduction of the oxygen-comprising gas into the reactor being throttled back or shut off at the end of each production step and the production step being continued until the oxygen concentration in the overhead stream decreases to 4.5% by volume, based on the total volume of the overhead stream.

When the desired concentration has been reached, the introduction of the starting gas mixture comprising the n-butenes and any remaining introduction of the oxygen-comprising gas are shut off and the regeneration step using the overhead stream from the absorption column as oxygen-comprising regeneration gas mixture or substream of the oxygen-comprising regeneration gas mixture commences.

If the oxygen concentration decreases during the course of the regeneration as a result of burning-off of carbonaceous material, it can be restored to the original concentration by addition of further oxygen-comprising gas. Furthermore, the oxygen concentration and the reactor temperature can be increased during the course of the regeneration.

The oxygen-comprising regeneration gas mixture comprises a proportion by volume of molecular oxygen of not more than 5%, preferably not more than 4.5%, at the beginning of the regeneration.

The oxygen-comprising regeneration gas mixture optionally additionally comprises inert gases, water vapor and/or hydrocarbons. As possible inert gases, mention may be made of nitrogen, argon, neon, helium, CO and CO₂. The amount of inert gases is in the case of nitrogen generally 90% by volume or less, preferably 85% by volume or less and more preferably 80% by volume or less. In the case of constituents other than nitrogen, it is generally 30% by volume or less, preferably 20% by volume or less.

Furthermore, water vapor can also be comprised in the oxygen-comprising regeneration gas mixture. Nitrogen is present to adjust the oxygen concentration, and the same applies to water vapor. Water vapor can also be present in order to remove the heat of reaction and as mild oxidant for the removal of carbon-comprising deposits. When water vapor is introduced into the reactor at the beginning of the regeneration, preference is given to introducing a proportion by volume of 0-50%, preferably 0-10% and more preferably 0.1-10%. The proportion of water vapor can be increased during the course of the regeneration. The amount of nitrogen is selected so that the proportion by volume of molecular oxygen in the regeneration gas mixture at the beginning of the regeneration is 20-99%, preferably 50-98% and even more preferably 60-96%. The proportion of nitrogen can be reduced during the course of the regeneration.

Furthermore, the oxygen-comprising regeneration gas mixture can comprise hydrocarbons and reaction products of the oxidative dehydrogenation. The proportion by volume of these substances in the oxygen-comprising regeneration gas mixture is generally less than 50%, preferably less than 10% and even more preferably less than 5%. The hydrocarbons can be saturated and unsaturated, branched and unbranched hydrocarbons such as methane, ethane, ethene, acetylene, propane, propene, propyne, n-butane, isobutane, n-butene, isobutene, n-pentane and also dienes such as 1,3-butadiene and 1,2-butadiene. In particular, they comprise hydrocarbons which do not display any reactivity in the presence of oxygen under the regeneration conditions in the presence of the catalyst.

During stable operation, the residence time in the reactor during the regeneration according to the present invention is not subject to any particular restrictions, but the lower limit is generally 0.5 s or more, preferably 1 s or more and more preferably 3 s or more. The upper limit is 4000.0 s or less, preferably 500.0 s or less and even more preferably 100.0 s or less. The ratio of throughput of mixed gas to the catalyst volume in the interior of the reactor is 1-7000 h⁻¹, preferably 7-3500 h⁻¹ and even more preferably 35-1500 h⁻¹.

### Catalyst

In the present process, a heterogeneous, particulate multimetal oxide catalyst comprising molybdenum and at least one further metal as active composition is used. Suitable catalysts are generally based on an Mo—Bi—O-comprising multimetal oxide system which generally additionally comprises iron. In general, the catalyst system comprises further additional components from groups 1 to 15 of the Periodic Table, for example potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

In general, the catalytically active multimetal oxide comprising molybdenum and at least one further metal has the general formula (I),

Mo₁₂BiₐFe_{b}Co_{c}Ni_{d}CrₑX¹_{f}X²_{g}Oₓ (I)

where the variables have the following meanings:
X¹═W, Sn, Mn, La, Ce, Ge, Ti, Zr, Hf, Nb, P, Si, Sb, Al, Cd and/or Mg;
X²═Li, Na, K, Cs and/or Rb,
a=0.1 to 7, preferably from 0.3 to 1.5;
b=0 to 5, preferably from 2 to 4;
c=0 to 10, preferably from 3 to 10;
d=0 to 10;
e=0 to 5, preferably from 0.1 to 2;
f=0 to 24, preferably from 0.1 to 2;
g=0 to 2, preferably from 0.01 to 1; and
x=a number which is determined by the valence and abundance of the elements other than oxygen in (I).

The catalyst can be an all-active catalyst or a coated catalyst. If it is a coated catalyst, it has a support body (a) and a shell (b) comprising the catalytically active multimetal oxide which comprises molybdenum and at least one further metal and has the general formula (I).

Support materials suitable for coated catalysts are, for example, porous or preferably nonporous aluminum oxides, silicon dioxide, zirconium dioxide, silicon carbide or silicates such as magnesium silicate or aluminum silicate (e.g. steatite of the type C 220 from CeramTec). The materials of the support bodies are chemically inert. The support materials can be porous or nonporous. The support material is preferably nonporous (ratio of the overall volume of the pores to the volume of the support body preferably ≦1%).

The use of essentially nonporous, spherical supports composed of steatite (e.g. steatite of the type C 220 from CeramTec) and having a rough surface and a diameter of from 1 to 8 mm, preferably from 2 to 6 mm, particularly preferably from 2 to 3 or from 4 to 5 mm, is particularly useful. However, the use of cylinders composed of chemically inert support material as support bodies, whose length is from 2 to 10 mm and whose external diameter is from 4 to 10 mm, is also useful. In the case of rings as support bodies, the wall thickness is also usually from 1 to 4 mm. Preferred ring-shaped support bodies have a length of from 2 to 6 mm, an external diameter of from 4 to 8 mm and a wall thickness of from 1 to 2 mm. Rings having the geometry 7 mm×3 mm×4 mm (external diameter x length x internal diameter) are particularly useful as support bodies. The layer thickness of the shell (b) of a multimetal oxide composition comprising molybdenum and at least one further metal is generally from 5 to 1000 μm. Preference is given to from 10 to 800 μm, particularly preferably from 50 to 600 μm and very particularly preferably from 80 to 500 μm.

Examples of Mo—Bi—Fe—O-comprising multimetal oxides are Mo—Bi—Fe—Cr—O— or Mo—Bi—Fe—Zr—O-comprising multimetal oxides. Preferred systems are described, for example, in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mo_{0.2}BiFe₃Co_{4.5}Ni_{2.5}Cr_{0.5}K_{0.1}Oₓ, Mo_{13.75}BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo_{0.2}BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and their preparation are also described in U.S. Pat. No. 4,423,281 (Mo₁₂BiNi₈Pb_{0.5}Cr₃K_{0.2}Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),

where
X¹═Si, Mn and/or Al,
X²═Li, Na, K, Cs and/or Rb,
0.2≦a≦1,
0.5≦b≦10,
0≦c≦10,
0≦d≦10,
2≦c+d≦10
0≦e≦2,
0≦f≦10
0≦g≦0.5
y=a number which is determined by the valence and abundance of the elements other than oxygen in (Ia) in order to achieve charge neutrality.

Preference is given to catalysts whose catalytically active oxide composition comprises only Co from among the two metals Co and Ni (d=0). X¹ is preferably Si and/or Mn and X² is preferably K, Na and/or Cs, with particular preference being given to X²═K.

The stoichiometric coefficient a in formula (Ia) is preferably such that 0.4≦a≦1, particularly preferably 0.4≦a≦0.95. The value of the variable b is preferably in the range 1≦b≦5 and particularly preferably in the range 2≦b≦4. The sum of the stoichiometric coefficients c+d is preferably in the range 4≦c+d≦8 and particularly preferably in the range 6≦c+d≦8. The stoichiometric coefficient e is preferably in the range 0.1≦e≦2 and particularly preferably in the range 0.2≦e≦1. The stoichiometric coefficient g is advantageously ≧0. Preference is given to 0.01≦g≦0.5 and particular preference is given to 0.05≦g≦0.2.

The value of the stoichiometric coefficient for oxygen, y, is determined by the valence and abundance of the cations in order to achieve charge neutrality. Coated catalysts having catalytically active oxide compositions whose molar ratio of Co/Ni is at least 2:1, preferably at least 3:1 and particularly preferably at least 4:1, are advantageous. It is best for only Co to be present.

The coated catalyst is produced by applying a layer comprising the multimetal oxide comprising molybdenum and at least one further metal to the support body by means of a binder, drying and calcining the coated support body.

Finely divided multimetal oxides comprising molybdenum and at least one further metal which are to be used can in principle be obtained by producing an intimate dry mixture of starting compounds of the elemental constituents of the catalytically active oxide composition and thermally treating the intimate dry mixture at a temperature of from 150 to 650° C.

### Oxidative Dehydrogenation (Oxydehydrogenation, ODH)

Embodiments of the process of the invention are described in detail below.

In a plurality of production steps (i), an oxidative dehydrogenation of n-butenes to 1,3-butadiene is carried out by mixing a starting gas mixture comprising n-butenes with an oxygen-comprising gas and optionally additional inert gas and/or water vapor and optionally a recycle stream. The gas mixture obtained is brought into contact with a catalyst arranged in a fixed bed of catalyst in a fixed-bed reactor at a temperature of from 330 to 490° C. The temperatures mentioned relate to the temperature of the heat transfer medium.

The reaction temperature of the oxydehydrogenation is generally controlled by means of a heat transfer medium. Possible liquid heat transfer media of this type are, for example melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is from 330 to 490° C., preferably from 350 to 450° C. and particularly preferably from 365 to 420° C.

Owing to the exothermic nature of the reactions which occur, the temperature in particular sections in the interior of the reactor during the reaction can be higher than that of the heat transfer medium and a hot spot is formed. The position and magnitude of the hot spot is determined by the reaction conditions, but can also be regulated via the dilution ratio of the catalyst bed or the flow of mixed gas. The difference between hot spot temperature and the temperature of the heat transfer medium is generally 1-150° C., preferably 10-100° C. and particularly preferably 20-80° C. The temperature at the end of the catalyst bed is generally 0-100° C. above, preferably 0.1-50° C. above, particularly preferably 1-25° C. above, the temperature of the heat transfer medium.

The oxydehydrogenation can be carried out in all fixed-bed reactors known from the prior art, for example in a tray oven, in a fixed-bed tube reactor or shell-and-tube reactor or in a plate heat exchanger. A shell-and-tube reactor is preferred.

The oxidative dehydrogenation is preferably carried out in fixed-bed tube reactors or fixed-bed shell-and-tube reactors. The reaction tubes (like the other elements of the shell-and-tube reactor) are generally made of steel. The wall thickness of the reaction tubes is typically from 1 to 3 mm. Its internal diameter is generally (uniformly) from 10 to 50 mm or from 15 to 40 mm, frequently from 20 to 30 mm. The number of reaction tubes accommodated in the shell-and-tube reactor is generally at least 1000, or 3000, or 5000, preferably at least 10 000. The number of reaction tubes accommodated in the shell-and-tube reactor is frequently from 15 000 to 30 000 or up to 40 000 or up to 50 000. The length of the reaction tubes is normally a few meters, with a reaction tube length in the range from 1 to 8 m, frequently from 2 to 7 m, often from 2.5 to 6 m, being typical.

Furthermore, the catalyst bed which is installed in the ODH reactor can consist of a single zone or of 2 or more zones. These zones can consist of pure catalyst or be diluted with a material which does not react with the starting gas or components of the product gas from the reaction. Furthermore, the catalyst zones can consist of all-active material and/or supported coated catalysts.

As starting gas, it is possible to use pure n-butenes (1-butene and/or cis/trans-2-butene) or else a gas mixture comprising n-butenes. Such a gas mixture can, for example, be obtained by nonoxidative dehydrogenation of n-butane. It is also possible to use a fraction which comprises n-butenes (1-butene and/or 2-butene) as main constituent and has been obtained from the C₄ fraction from a naphtha cracker by separating off 1,3-butadiene and isobutene. Furthermore, it is also possible to use, as starting gas, gas mixtures which comprise pure 1-butene, cis-2-butene, trans-2-butene or mixtures thereof and have been obtained by dimerization of ethylene. In addition, gas mixtures which comprise n-butenes and have been obtained by fluid catalytic cracking (FCC) can also be used as starting gas.

In an embodiment of the process of the invention, the starting gas mixture comprising n-butenes is obtained by nonoxidative dehydrogenation of n-butane. The coupling of a nonoxidative catalytic dehydrogenation with the oxidative dehydrogenation of the n-butenes formed makes it possible to obtain a high yield of 1,3-butadiene, based on n-butane used. The nonoxidative catalytic dehydrogenation of n-butane gives a gas mixture comprising secondary constituents in addition to 1,3-butadiene, 1-butene, 2-butene and unreacted n-butane. Usual secondary constituents are hydrogen, water vapor, nitrogen, CO and CO₂, methane, ethane, ethene, propane and propene. The composition of the gas mixture leaving the first dehydrogenation zone can vary greatly depending on the mode of operation of the dehydrogenation. Thus, when the dehydrogenation is carried out with introduction of oxygen and additional hydrogen, the product gas mixture has a comparatively high content of water vapor and carbon oxides. In the case of a mode of operation without introduction of oxygen, the product gas mixture from the nonoxidative dehydrogenation has a comparatively high content of hydrogen.

The product gas stream from the nonoxidative dehydrogenation of n-butane typically comprises from 0.1 to 15% by volume of 1,3-butadiene, from 1 to 15% by volume of 1-butene, from 1 to 25% by volume of 2-butene (cis/trans-2-butene), from 20 to 70% by volume of n-butane, from 1 to 70% by volume of water vapor, from 0 to 10% by volume of low-boiling hydrocarbons (methane, ethane, ethene, propane and propene), from 0.1 to 40% by volume of hydrogen, from 0 to 70% by volume of nitrogen and from 0 to 5% by volume of carbon oxides. The product gas stream from the nonoxidative dehydrogenation can be fed without further work-up to the oxidative dehydrogenation.

Furthermore, any impurities in a range in which the effectiveness of the present invention is not inhibited can be present in the starting gas for the oxydehydrogenation. In the preparation of 1,3-butadiene from n-butenes (1-butene and cis/trans-2-butene), saturated and unsaturated, branched and unbranched hydrocarbons such as methane, ethane, ethene, acetylene, propane, propene, propyne, n-butane, isobutane, isobutene, n-pentane and also dienes such as 1,2-butadiene may be mentioned as impurities. The amounts of impurities are generally 70% or less, preferably 50% or less, more preferably 40% or less and particularly preferably 30% or less. The concentration of linear monoolefins having 4 or more carbon atoms (n-butenes and higher homologues) in the starting gas is not subject to any particular restrictions; it is generally 35.00-99.99% by volume, preferably 50.0-99.0% by volume and even more preferably 60.0-95.0% by volume.

To carry out the oxidative dehydrogenation with full conversion of n-butenes, a gas mixture having a molar oxygen: n-butene ratio of at least 0.5 is required. Preference is given to working at a molar oxygen: n-butene ratio of from 0.55 to 10. To set this value, the starting material gas can be mixed with oxygen or an oxygen-comprising gas, for example air, and optionally additional inert gas and/or steam. The oxygen-comprising gas mixture obtained is then fed to the oxydehydrogenation.

The gas comprising molecular oxygen is a gas which generally comprises more than 10% by volume, preferably more than 15% by volume and even more preferably more than 20% by volume, of molecular oxygen and is specifically preferably air. The upper limit to the content of molecular oxygen is generally 50% by volume or less, preferably 30% by volume or less and even more preferably 25% by volume or less. In addition, any inert gases in a range in which the effectiveness of the present invention is not inhibited can be present in the gas comprising molecular oxygen. As possible inert gases, mention may be made of nitrogen, argon, neon, helium, CO, CO₂ and water. The amount of inert gases is in the case of nitrogen generally 90% by volume or less, preferably 85% by volume or less and even more preferably 80% by volume or less. In the case of constituents other than nitrogen, the amount is generally 10% by volume or less, preferably 1% by volume or less. If this amount becomes too large, it becomes increasingly more difficult to supply the reaction with the necessary oxygen.

Furthermore, inert gases such as nitrogen and also water (as water vapor) can be comprised together in the mixed gas composed of starting gas and the gas comprising molecular oxygen. Nitrogen serves to set the oxygen concentration and to prevent formation of an explosive gas mixture, and the same applies to water vapor. Water vapor also serves to control carbonization of the catalyst and to remove the heat of reaction. Preference is given to mixing water (as water vapor) and nitrogen into the mixed gas and introducing this into the reactor. When water vapor is introduced into the reactor, it is preferably introduced in a proportion of 0.01-5.0 (parts by volume), preferably 0.1-3 and even more preferably 0.2-2, based on the amount of the abovementioned starting gas introduced. When nitrogen gas is introduced into the reactor, it is preferably introduced in a proportion of 0.1-8.0 (parts by volume), preferably 0.5-5.0 and even more preferably 0.8-3.0, based on the amount of the abovementioned starting gas introduced.

The proportion of the hydrocarbon-comprising starting gas in the mixed gas is generally 4.0% by volume or more, preferably 5.0% by volume or more and even more preferably 6.0% by volume or more. On the other hand, the upper limit is 20% by volume or less, preferably 15.0% by volume or less and even more preferably 12.0% by volume or less. To reliably avoid the formation of explosive gas mixtures, before the mixed gas is obtained, nitrogen gas is firstly introduced into the starting gas or into the gas comprising molecular oxygen, the starting gas and the gas comprising molecular oxygen are mixed so as to give the mixed gas and this mixed gas is then preferably introduced.

During stable operation, the residence time in the production mode in the present invention is not subject to any particular restrictions, but the lower limit is generally 0.3 s or more, preferably 0.7 s or more and even more preferably 1.0 s or more. The upper limit is 5.0 s or less, preferably 3.5 s or less and even more preferably 2.5 s or less. The ratio of flow of mixed gas to the amount of catalyst in the interior of the reactor is 500-8000 h⁻¹, preferably 800-4000 h⁻¹ and even more preferably 1200-3500 h⁻¹. The n-butene load over the catalyst (expressed in g_{butenes}/(g_{catalyst}*hour) in stable operation is generally 0.1-5.0 h⁻¹, preferably 0.2-3.0 h⁻¹, and even more preferably 0.25-1.0 h⁻¹. Volume and mass of the catalyst relate to the complete catalyst consisting of support and active composition.

### Work-Up of the Product Gas Stream

The product gas stream leaving the oxidative dehydrogenation generally comprises unreacted 1-butene and 2-butene, oxygen and water vapor in addition to 1,3-butadiene. As secondary components, it generally further comprises carbon monoxide, carbon dioxide, inert gases (mainly nitrogen), low-boiling hydrocarbons such as methane, ethane, ethene, propane and propene, butane and isobutane, possibly hydrogen and also possibly oxygen-comprising hydrocarbons, known as oxygenates. Oxygenates can, for example, be formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

For example, the product gas stream leaving the oxidative dehydrogenation can comprise from 1 to 40% by volume of 1,3-butadiene, from 20 to 80% by volume of n-butane, from 0 to 5% by volume of isobutane, from 0.5 to 40% by volume of 2-butene, from 0 to 5% by volume of 1-butene, from 0 to 70% by volume of water vapor, from 0 to 10% by volume of low-boiling hydrocarbons (methane, ethane, ethene, propane and propene), from 0 to 40% by volume of hydrogen, from 0 to 30% by volume of oxygen, from 0 to 70% by volume of nitrogen, from 0 to 10% by volume of carbon oxides and from 0 to 10% by volume of oxygenates.

Some of the oxygenates can oligomerize and dehydrogenate further on the catalyst surface and in the work-up and form deposits comprising carbon, hydrogen and oxygen, hereinafter referred to as carbonaceous material. These deposits can, for the purpose of cleaning and regeneration, lead to interruptions in operation of the process and are therefore undesirable. Typical carbonaceous material precursors comprise styrene, fluorenone and anthraquinone.

The product gas stream at the output from the reactor has a temperature close to the temperature at the end of the catalyst bed. The product gas stream is then brought to a temperature of 150-400° C., preferably 160-300° C., particularly preferably 170-250° C. It is possible to insulate the line through which the product gas stream flows in order to keep the temperature in the desired range, but the use of a heat exchanger is preferred. This heat exchanger system can be of any type as long as this system enables the temperature of the product gas to be kept at the desired level. As examples of a heat exchanger, mention may be made of helical heat exchangers, plate heat exchangers, double-walled heat exchangers, multitube heat exchangers, boiler-helix heat exchangers, boiler-jacket heat exchangers, liquid-liquid contact heat exchangers, air heat exchangers, direct contact heat exchangers and finned tube heat exchangers. Since, while the temperature of the product gas is brought to the desired temperature, part of the high-boiling by-products comprised in the product gas can precipitate, the heat exchanger system should preferably have two or more heat exchangers. If two or more heat exchangers provided are arranged in parallel and distributed cooling of the product gas obtained is thereby made possible in the heat exchangers, the amount of high-boiling by-products which deposit in the heat exchanger decreases and the operating time of the heat exchangers can be extended in this way. As an alternative to the abovementioned method, the two or more heat exchangers provided can be arranged in parallel. The product gas is fed to one or more, but not all, of the heat exchangers and, after a particular period of operation, these heat exchangers are relieved by other heat exchangers. In this method, cooling can be continued, part of the heat of reaction can be recovered and, in parallel thereto, the high-boiling by-products deposited in one of the heat exchangers can be removed. As an organic solvent as described above, it is possible to use any solvent as long as it is able to dissolve the high-boiling by-products; for example, it is possible to use an aromatic hydrocarbon solvent such as toluene, xylene, etc., or an alkaline aqueous solvent, e.g. the aqueous solution of sodium hydroxide, for this purpose.

As intermediate step, the product gas stream is then preferably fed to a quench in which the predominant part, i.e. at least 55% by volume, of the high-boiling hydrocarbons, i.e. hydrocarbons having a boiling point of 95° C. or above at one atmosphere, and also part of the water are separated off by the bottom stream by direct contacting with a cooling medium to give a side stream which is fed as such or via a compressor into the absorption column. The quench can consist of only one stage or of a plurality of stages. The product gas stream is thus brought into direct contact with the one coolant and cooled thereby. As coolant, it is possible to use water or aqueous solutions. Preference is given to using organic solvents, in particular aromatic hydrocarbons.

In general, the product gas has, depending on the presence and temperature level of a heat exchanger, a temperature of 10-440° C. before the quench. The product gas is brought into contact with the coolant in the quenching stage. In this case the coolant can be introduced by a nozzle in order to achieve very efficient mixing with the product gas. For the same purpose, it is possible to install internals, for example further nozzles, which pass both the product gas and the coolant in the quenching stage. The coolant inlet into the quench is designed so that blockage by deposits in the region of the coolant inlet is minimized.

Since the loading of the coolant with secondary components increases over the course of time, part of the loaded coolant can be taken off from the circuit as purge stream and the amount being circulated can be kept constant by addition of coolant which is not loaded. The ratio of amount discharged to amount added depends, inter alia, on the choice of the coolant, the loading of water vapor in the product gas and the product gas temperature at the end of the first quenching stage.

Depending on temperature, pressure, water content of the product gas and choice of the coolant, condensation of water can occur in the quenching stage. In this case, an additional aqueous phase which can additionally comprise water-soluble secondary components can be formed. This can then be taken off at the bottom of the quenching stage.

In general, the product gas is cooled to from 5 to 100° C., preferably to 15-85° C. and even more preferably to 30-70° C., up to the gas outlet from the quenching stage. The pressure in the quenching stage is not subject to any particular restrictions, but is generally 0.01-4 bar gauge, preferably 0.1-2 bar gauge and particularly preferably 0.2-1 bar gauge.

To minimize the entrainment of liquid constituents from the quench into the offgas line, it is possible to undertake suitable constructional measures, for example the installation of a demister. Furthermore, high-boiling substances which are not separated off from the product gas in the quench can be removed from the product gas by means of further constructional measures, for example further gas scrubs.

The quench gives a product gas stream which comprises n-butane, 1-butene, 2-butenes, 1,3-butadiene, possibly oxygen, hydrogen, water vapor, small amounts of methane, ethane, ethene, propane and propene, isobutane, carbon oxides, inert gases and amounts of the coolant used in the quench. Furthermore, traces of high-boiling components which have not been quantitatively separated off in the quench can remain in this gas stream.

The product gas stream from the quench is then compressed preferably in at least one first compression stage and subsequently cooled, with at least one condensate stream comprising water being condensed out and a gas stream comprising n-butane, 1-butene, 2-butenes, 1,3-butadiene, possibly hydrogen, water vapor, small amounts of methane, ethane, ethene, propane and propene, isobutane, carbon oxides and inert gases, possibly oxygen and hydrogen remaining. The compression can be carried out in one or more stages. Overall, the gas stream is compressed from a pressure in the range from 1.0 to 4.0 bar absolute to a pressure in the range from 3.5 to 20 bar absolute. Each compression stage is followed by a cooling stage in which the gas stream is cooled to a temperature in the range from 15 to 60° C. The condensate stream can thus also comprise a plurality of streams in the case of multistage compression. The condensate stream generally comprises at least 50% by weight, preferably at least 70% by weight, of water and additionally comprises small amounts of low boilers, C4-hydrocarbons, oxygenates and carbon oxides.

Suitable compressors are, for example, turbocompressors, rotary piston compressors and reciprocating piston compressors. The compressors can, for example, be driven by means of an electric motor, an expander or a gas or steam turbine. Typical compression ratios (entry pressure: exit pressure) per compression stage are, depending on the construction type, in the range from 1.5 to 3.0. Cooling of the compressed gas is carried out by means of heat exchangers which can be configured, for example, as shell-tube, helical or plate heat exchangers. Cooling water or heat transfer oils can be used as coolant in the heat exchangers. In addition, preference is given to using air cooling using blowers.

As an alternative, the product gas stream from the fixed-bed reactor can, as intermediate step, be fed directly to a compression stage as described above and in this brought to a pressure of from 3.5 to 20 bar absolute.

In the following step, incondensable or low-boiling gas constituents comprising oxygen, low-boiling hydrocarbons (methane, ethane, ethene, propane, propene), carbon oxides and inert gases are separated off from the process gas stream by absorption of the C₄-hydrocarbons in a high-boiling absorption medium in an absorption column as gas stream and subsequent desorption of the C₄-hydrocarbons. This step preferably comprises the substeps a) to c):
a) absorption of the C₄-hydrocarbons comprising 1,3-butadiene and n-butenes in a high-boiling absorption medium to give an absorption medium stream loaded with Ca-hydrocarbons and an overhead stream comprising oxygen, low-boiling hydrocarbons, i.e. hydrocarbons having a boiling point of less than 95° C. at one atmosphere, residues of C₄-hydrocarbons, residues of high-boiling hydrocarbons, i.e. hydrocarbons having a boiling point of 95° C. or above at one atmosphere, optionally inert gases, in particular nitrogen, optionally carbon oxides and optionally water vapor,
b) removal of oxygen from the absorption medium stream loaded with C₄-hydrocarbons from substep a) by stripping with an incondensable gas stream to give an absorption medium stream loaded with C₄-hydrocarbons and
c) desorption of the Ca-hydrocarbons from the loaded absorption medium stream to give a C₄ product gas stream consisting essentially of C₄-hydrocarbons.

For this purpose, the compressed product gas stream is brought into contact with an inert absorption medium in the absorption stage and the predominant part of the Ca-hydrocarbons are absorbed in the inert absorption medium to give an absorption medium loaded with Ca-hydrocarbons and an overhead stream comprising the remaining gas constituents. In a desorption stage, the C₄-hydrocarbons are liberated again from the high-boiling absorption medium.

The absorption stage can be carried out in any suitable absorption column known to those skilled in the art. Absorption can be effected by simple passing of the product gas stream through the absorption medium. However, it can also be carried out in columns or in rotary absorbers. This can be carried out in concurrent, countercurrent or cross-current. The absorption is preferably carried out in countercurrent. Suitable absorption columns are, for example, tray columns having bubble cap trays, centrifugal trays and/or sieve trays, columns having structured packings, e.g. sheet metal packings having a specific surface area of from 100 to 1000 m²/m³, e.g. Mellapak® 250 Y, and columns packed with random packing elements. However, trickle towers and spray towers, graphite block absorbers, surface absorbers such as thick film and thin film absorbers and also rotary columns, plate scrubbers, crossed-spray scrubbers and rotary scrubbers are also possible.

In a preferred embodiment, the gas stream comprising 1,3-butadiene, n-butenes and the low-boiling and uncondensable gas constituents is fed into the lower region of an absorption column. The high-boiling absorption medium is introduced in the upper region of the absorption column.

High-boiling absorption media used in the absorption stage are generally high-boiling nonpolar solvents in which the C₄-hydrocarbon mixture to be separated off has a significantly higher solubility than do the remaining gas constituents to be separated off. Suitable absorption media are comparatively nonpolar organic solvents, for example aliphatic C₈-C₁₈-alkanes, or aromatic hydrocarbons such as the middle oil fractions from paraffin distillation, toluene or ethers having bulky groups, or mixtures of these solvents, with a polar solvent such as 1,2-dimethyl phthalate being able to be added to these. Further suitable absorption media are esters of benzoic acid and phthalic acid with straight-chain C₁-C₈-alkanols, and also heat transfer oils such as biphenyl and diphenyl ether, chloro derivatives thereof and also triarylalkenes. One suitable absorption medium is a mixture of biphenyl and diphenyl ether, preferably in the azeotropic composition, for example the commercially available Diphyl®. This solvent mixture frequently comprises dimethyl phthalate in an amount of from 0.1 to 25% by weight.

In a preferred embodiment, the same solvent as in the quench is used in the absorption stage.

An offgas stream comprising essentially oxygen, low-boiling hydrocarbons (methane, ethane, ethene, propane, propene), possibly C₄-hydrocarbons (butane, n-butenes, 1,3-butadiene), optionally inert gases, optionally carbon oxides and optionally water vapor is taken off at the top of the absorption column. This stream can be partly fed to the fixed bed reactor. This enables, for example, the inlet stream to the fixed bed reactor to be adjusted to the desired Ca-hydrocarbon content.

At the bottom of the absorption column, residues of oxygen dissolved in the absorption medium are discharged into a further column by the flushing with a gas. Stripping-out of the oxygen in step b) can be carried out in any suitable column known to those skilled in the art. Stripping can be effected by simple passing of incondensable gases, preferably inert gases such as nitrogen, through the loaded absorption solution. In the upper part of the absorption column, C4 stripped out is washed back into the absorption solution by feeding the gas stream back into this absorption column. This can be effected both by pipe connection of the stripper column or by direct installation of the stripper column below the absorber column. Since the pressure in the stripper column part and the absorption column part is the same, according to the invention, this direct coupling can be effected. Suitable stripping columns are, for example, tray columns having bubble cap trays, centrifugal trays and/or sieve trays, columns having structured packing, e.g. sheet metal packings having a specific surface area of from 100 to 1000 m²/m³, e.g. Mellapak® 250 Y, and columns packed with random packing elements. However, trickle towers and spray towers and also rotary columns, plate scrubbers, crossed-spray scrubbers and rotary scrubbers are also possible. Suitable gases are, for example, nitrogen or methane.

The absorption medium stream loaded with C₄-hydrocarbons comprises water. This is separated off as stream from the absorption medium in a decanter so as to give a stream which now comprises only the water dissolved in the absorption medium.

The loaded absorption medium stream can be worked-up further in any known way, in particular by desorption and subsequent extractive distillation.

### Regeneration of the Multimetal Oxide Catalyst

According to the present process, a regeneration step (ii) is carried out between each two production steps (i). The reactor can be operated in the production mode (i) until, for example, catalyst deactivation has reached a particular, prescribed value and, for example, the conversion at a constant reaction temperature has dropped by 20%, preferably 10%, more preferably 5% and particularly preferably 2%. Furthermore, the reactor can be operated in the production step until the pressure drop over the reactor has increased by a particular, prescribed value, for example by 1000 mbar, preferably 500 mbar, more preferably 100 mbar and particularly preferably 20 mbar, or until a particular, prescribed duration of the production step has elapsed, for example 2000 h, preferably 1000 h, more preferably 500 h and particularly preferably 340 h.

In the process of the invention for the oxidative dehydrogenation of n-butenes to 1,3-butadiene, an economical regeneration gas, which comprises less than 5% by volume, preferably less than 4.5% by volume, of oxygen, without going through the explosive range, is generated.

The measurement of the proportion of oxygen in the regeneration gas can be carried out by any method known to those skilled in the art, in particular gas-chromatographically, spectroscopically, paramagnetically or electrochemically.

If the oxygen concentration of the recycle gas decreases during the course of the regeneration due to burning-off of carbonaceous material, it can be brought back to the original concentration by addition of oxygen-comprising gas.

Both the oxygen concentration in the recycle gas and also the temperature can be increased during the course of the regeneration.

The invention is illustrated below with the aid of a drawing and examples:

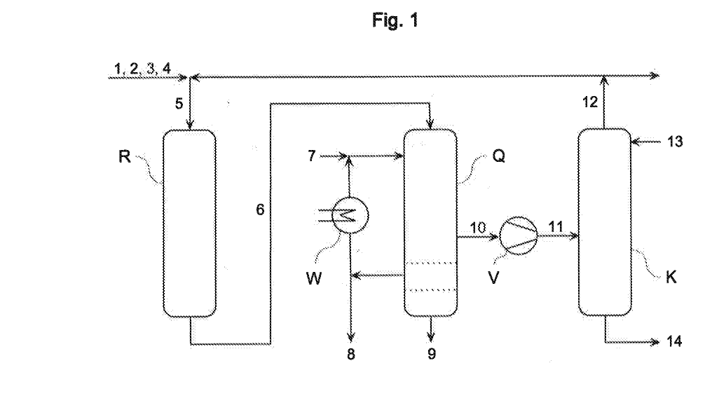

FIG. 1, the only FIGURE, shows a preferred embodiment of a plant for carrying out the process of the invention.

A stream 5 which, in the preferred embodiment depicted, is obtained by mixing a starting gas mixture 1 comprising the n-butenes with an oxygen-comprising gas 2, an inert gas stream 3 and a stream 4 comprising water vapor is fed to the reactor R. At the lower end of the reactor R, a product gas stream 6 is taken off and fed to a quench Q in the upper part thereof and quenched by means of a coolant, stream 7, to give a bottom stream 9 and a side stream 10 which is fed via a compressor V as compressed stream 11 to an absorption column K. From the quench Q, a coolant stream 8 is taken off and partly discharged while the remainder is fed back via a heat exchanger W to the quench.

A high-boiling absorption medium, stream 13, is introduced into the absorption column K in the upper region thereof and a loaded absorption medium stream 14 is taken off at the bottom. An overhead stream 12 is taken off from the absorption column K and is partly discharged while the remainder is recycled to the reactor R.

### EXAMPLES

A salt bath reactor R having 24 000 horizontal tubes which are composed of stainless steel 1.4571, have a length of 5 m and an internal diameter of 29.7 mm and are filled with 2550 ml of a multimetal oxide catalyst whose production is described below is used.

### Catalyst Production

Two solutions A and B were produced.

### Solution A:

3200 g of water were placed in a 10 l stainless steel pot. While stirring by means of an anchor stirrer, 5.2 g of a KOH solution (32% by weight of KOH) were added to the initially charged water. The solution was heated to 60° C. 1066 g of an ammonium heptamolybdate solution ((NH4)6Mo7O24*4 H20, 54% by weight of Mo) were then added a little at a time over a period of 10 minutes. The suspension obtained was stirred for another 10 minutes.

### Solution B:

1771 g of a cobalt(II) nitrate solution (12.3% by weight of Co) were placed in a 5 l stainless steel pot and heated to 60° C. while stirring (anchor stirrer). 645 g of an iron(III) nitrate solution (13.7% by weight of Fe) were then added a little at a time over a period of 10 minutes while maintaining the temperature. The solution formed was stirred for another 10 minutes. 619 g of a bismuth nitrate solution (10.7% by weight of Bi) were then added while maintaining the temperature. After stirring for a further 10 minutes, 109 g of chromium(III) nitrate were added a lithe at a time in solid form and the dark red solution formed was stirred for another 10 minutes.

While maintaining the 60° C., the solution B was pumped by means of a peristaltic pump into solution A over a period of 15 minutes. During the addition and afterwards, the mixture was stirred by means of a high-speed stirrer (Ultra-Turrax). After the addition was complete, stirring was continued for another 5 minutes.

The suspension obtained was spray dried in a spray dryer from NIRO (spray head No. FOA1, speed of rotation 25 000 rpm) over a period of 1.5 hours. The reservoir temperature was maintained at 60° C. during this time. The gas inlet temperature of the spray dryer was 300° C., and the gas outlet temperature was 110° C. The powder obtained had a particle size (d50) of less than 40 μm.

The powder obtained was mixed with 1% by weight of graphite, compacted twice under a pressing pressure of 9 bar and broken up through a sieve having a mesh opening of 0.8 mm. The crushed material was in turn mixed with 2% by weight of graphite and the mixture was pressed by means of a Kilian S100 tableting press to give rings having dimensions of 5×3×2 mm (external diameter x length x internal diameter).

The catalyst precursor obtained was calcined batchwise (500 g) in a convection oven from Heraeus, DE (model K, 750/2 S, internal volume 55 l). The following program was used for this purpose:
heat over 72 minutes to 130° C., hold for 72 minutes
heat over 36 minutes to 190° C., hold for 72 minutes
heat over 36 minutes to 220° C., hold for 72 minutes
heat over 36 minutes to 265° C., hold for 72 minutes
heat over 93 minutes to 380° C., hold for 187 minutes
heat over 93 minutes to 430° C., hold for 187 minutes
heat over 93 minutes to 490° C., hold for 467 minutes

After the calcination, the catalyst having the calculated stoichiometry Mo12Co7Fe3Bi0.6K0.08Cr0.5 Ox was obtained.

The calcined rings were milled to a powder. Support bodies (steatite rings having dimensions of 7×3×4 mm (external diameter×height×internal diameter) were coated with the powder. For this purpose, 900 g in each case of the support were placed three times in a coating drum (2 l internal volume, angle of inclination of the drum central axis relative to the horizontal=)30°. The drum was set into rotation (36 rpm). About 70 ml of liquid binder (1:3 mixture of glycerol:water) were spayed onto the support (spraying air 200 standard I/h) via an atomizer nozzle operated using compressed air over a period of about 45 minutes. The nozzle was installed in such a way that the spray cone wetted the support bodies conveyed in the drum in the upper half of the rolling-down section. 230 g of the finely pulverulent precursor composition of the milled catalyst were in each case introduced via a powder screw into the drum, with the point of powder addition being located within the rolling-down section but below the spray cone. The powder addition was metered in such a way that a uniform distribution of the powder on the surface was obtained. After coating was complete, the resulting coated catalyst composed of precursor composition and the support body was dried at 300° C. in a drying oven for 4 hours.

Above the catalyst bed, there is an inert bed of shaped steatite bodies having a length of 60 cm. In eight of the tubes, known as thermotubes, there is in the center in each case a thermocouple sheath (external diameter 6 mm) having internal thermocouples in order to measure the temperature profile in the bed. A salt melt is passed around the tube in order to keep the exterior wall temperature constant. The temperature of the salt melt is 380° C.

The reaction gas 5 consists of an n-butene/n-butane stream composed of 20 mol % of n-butane and 80 mol % of n-butenes 1, steam 4, air 2 and recycled recycle gas. The recycled recycle gas is obtained from part of the overhead stream 12. The reaction gas 5 is firstly heated to 210° C. in a plate heat exchanger and then introduced from above into the reactor R. The reaction gas is heated to the salt bath temperature in the inert bed and reacted over the catalyst.

The product gas stream 6 obtained from the reactor R is cooled to 45° C. in a cooling tower Q, with the high-boiling secondary components being separated off. The side stream 10 obtained is compressed to 10 bar gauge in a compressor V and cooled back down to 45° C., with a condensate stream being discharged. The compressed and cooled stream 11 is then introduced into an absorption column K. In the absorption column K, n-butanes/n-butenes and 1,3-butadiene are dissolved in mesitylene as absorption medium 13 and passed as loaded solvent stream 14 to the further work-up. The remaining gas stream 12 is taken off at the top of the absorption column K and partly fed as recycled recycle gas stream to the reactor R.

### Example 1

The reactor R is supplied with 26.6 t/h of an n-butene/n-butane stream 1 consisting of 20 mol % of n-butane and 80 mol % of n-butenes, plus 4.32 t/h of steam 4 and 52 t/h of air 2 and 66 t/h of recycled recycle gas. 84% of the n-butenes are reacted in the reactor R and the yield of 1,3-butadiene is 76%. The oxygen concentration in the recycled recycle gas is 7.6 mol %. The gas phase at the reactor inlet thus comprises 8 mol % of n-butenes, 11.5 mol % of oxygen, 5 mol % of steam and also N₂ and small proportions of CO, CO₂ and argon. The average residence time of the gas in the work-up is 27 s under these conditions.

Over a period of 30 s, the air stream 2 is continuously reduced from 100% to 0% of the original amount. The gas introduced at the reactor inlet then comprises 12.5 mol % of n-butenes, 5.2 mol % of oxygen and about 7.8 mol % of steam. After a further 20 s, the n-butene/n-butane stream 1 is reduced from 100% to 0% of the original load and the regeneration commences. The mass throughput of the recycled recycle gas stream remains unchanged during this entire time. The composition at the reactor inlet is then about 0 mol % of n-butenes, 4.3 mol % of oxygen and about 10 mol % of steam. The steam stream 2 is shut off over the further 60 s.

The concentration of oxygen in the stream 12 and thus in the recycled recycle gas, the butene conversion in the reactor R and the magnitude of the hot spot as a function of time are shown in the following table:

| Time | Butene conversion | O₂ in stream 12 | Hot spot |
|---|---|---|---|
| (s) | [%] | [mol %] | [K] |
| 0 | 84 | 7.6 | 37 |
| 30 | 61 | 6.7 | 36 |
| 50 | — | 4.8 | 32 |
| 150 | — | 3.5 | 13 |

The air stream 3 is subsequently regulated so that the oxygen concentration in the stream 12 is 3-4 mol %. The recycle stream remains unchanged at a mass flow of about 66 t/h over the entire time by the remaining stream 12 being discharged.

During the entire regeneration, no explosive atmosphere is formed in the process and the temperatures measured in the thermotubes due to burning off of carbonaceous material in the catalyst bed are at no time more than 32° C. above the salt bath temperature.

### Example 2

The reactor R is supplied with 26.6 t/h of an n-butene/n-butane stream 1 consisting of 20 mol % of n-butane and 80 mol % of n-butenes, plus 4.32 t/h of steam 4 and 52 t/h of air 2 and 66 t/h of recycled recycle gas. 84% of the n-butenes are reacted in the reactor R and the yield of 1,3-butadiene is 76%. The oxygen concentration in the recycled recycle gas is 7.6 mol %. The gas phase at the reactor inlet thus comprises 8 mol % of n-butenes, 11.5 mol % of oxygen, 5 mol % of steam and also N2 and small proportions of CO, CO₂ and argon. The average residence time of the gas in the work-up is 27 s under these conditions.

Over a period of 60 s, the air stream 2 is continuously reduced from 100% to 0% of the original amount. The gas introduced at the reactor inlet then comprises 12.5 mol % of n-butenes, 3.6 mol % of oxygen and about 7.8 mol % of steam. After a further 10 s, the n-butene/n-butane stream 1 is reduced from 100% to 0% of the original load and the regeneration commences. The mass throughput of the recycled recycle gas stream remains unchanged during this entire time. The composition at the reactor inlet is then about 0 mol % of n-butenes, 3.5 mol % of oxygen and about 9.2 mol % of steam. The steam stream 2 is shut off over the further 60 s.

The concentration of oxygen in the stream 12 and thus in the recycled recycle gas, the n-butene conversion in the reactor R and the magnitude of the hot spot as a function of time are shown in the following table:

| Time | Butene conversion | O₂ in stream 12 | Hot spot |
|---|---|---|---|
| (s) | [%] | [mol %] | [K] |
| 0 | 84 | 7.6 | 37 |
| 60 | 47 | 4.7 | 35 |
| 70 | — | 3.9 | 32 |
| 130 | — | 3.4 | 14 |

The air stream 3 is subsequently regulated so that the oxygen concentration in the stream 12 is 3-4 mol %. The recycle stream remains unchanged at a mass flow of about 66 t/h over the entire time by the remaining stream 12 being discharged.

During the entire regeneration, no explosive atmosphere is formed in the process and the temperatures measured in the thermotubes due to burning off of carbonaceous material in the catalyst bed are at no time more than 32° C. above the salt bath temperature.

### Example 3

The reactor R is supplied with 26.6 t/h of an n-butene/n-butane stream 1 consisting of 20 mol % of n-butane and 80 mol % of n-butenes, plus 4.32 t/h of steam 4 and 52 t/h of air 2 and 66 t/h of recycled recycle gas. 84% of the n-butenes are reacted in the reactor R and the yield of 1,3-butadiene is 76%. The oxygen concentration in the recycled recycle gas is 7.6 mol %. The gas phase at the reactor inlet thus comprises 8 mol % of n-butenes, 11.5 mol % of oxygen, 5 mol % of steam and also N₂ and small proportions of CO, CO₂ and argon. The average residence time of the gas in the work-up is 27 s under these conditions.

Over a period of 30 s, the air stream 2 is continuously reduced from 100% to 0% of the original amount. The gas introduced at the reactor inlet then comprises 12.5 mol % of n-butenes, 5.2 mol % of oxygen and about 7.8 mol % of steam. After a further 60 s, the n-butene/n-butane stream 1 is reduced from 100% to 0% of the original load and the regeneration commences. The mass throughput of the recycled recycle gas stream remains unchanged during this entire time. The composition at the reactor inlet is then about 0 mol % of n-butenes, 1.5 mol % of oxygen and about 10 mol % of steam. The steam stream 2 is shut off over the further 60 s.

The concentration of oxygen in the stream 12 and thus in the recycled recycle gas, the n-butene conversion in the reactor R and the magnitude of the hot spot as a function of time are shown in the following table:

| Time | Butene conversion | O₂ in stream 12 | Hot spot |
|---|---|---|---|
| (s) | [%] | [mol %] | [K] |
| 0 | 84 | 7.6 | 37 |
| 30 | 61 | 6.7 | 36 |
| 90 | — | 1.7 | 21 |
| 150 | — | 1.5 | 5 |

The air stream 3 is subsequently regulated so that the oxygen concentration in the stream 12 is 2-3 mol %. The recycle stream remains unchanged at a mass flow of about 66 t/h over the entire time by the remaining stream 12 being discharged. During the entire regeneration, no explosive atmosphere is formed in the process and the temperatures measured in the thermotubes due to burning off of carbonaceous material in the catalyst bed are at no time more than 21° C. above the salt bath temperature.

### Comparative Example

The reactor R is supplied with 26.6 t/h of an n-butene/n-butane stream 1 consisting of 20 mol % of n-butane and 80 mol % of n-butenes, plus 4.32 t/h of steam 4 and 52 t/h of air 2 and 66 t/h of recycled recycle gas. 84% of the n-butenes are reacted in the reactor and the yield of 1,3-butadiene is 76%. The oxygen concentration in the recycled recycle gas is 7.6 mol %. The gas phase at the reactor inlet thus comprises 8 mol % of n-butenes, 11.5 mol % of oxygen, 5 mol % of steam and also N₂ and small proportions of CO, CO₂ and argon.

The air stream 2 and the n-butene/n-butane stream 1 are simultaneously shut off over a period of 30 s and regeneration commences. The gas introduced at the reactor inlet comprises 0 mol % of butenes, 6.8 mol % of oxygen and about 9.5 mol % of steam at this point in time. The steam stream 4 is then shut off over a further 60 s.

The course of the oxygen concentration in the overhead stream 12 and the course of the hot spot in the reactors R are shown in the following table.

| Time | Butene conversion | O₂ in stream 12 | Hot spot |
|---|---|---|---|
| (s) | [%] | [mol %] | [K] |
| 0 | 84 | 7.6 | 37 |
| 30 | — | 7.5 | 31 |
| 90 | — | 7.3 | 13 |
| 150 | — | 7.3 | 5 |

During the overall regeneration, the composition of the gas atmosphere in some regions of the work-up comes close to an explosive atmosphere. Furthermore, the temperatures measured in the thermotubes at the beginning of the regeneration are 31° C. above the salt bath temperature and the oxygen concentration at the reactor inlet is 6.8% by volume. Under these conditions, uncontrolled rapid burning-off of the carbonaceous material and destruction of the catalyst and/or the reactor R can easily occur.

1.-7. (canceled)

8. A process for the oxidative dehydrogenation of n-butenes to 1,3-butadiene in a fixed-bed reactor (R), which comprises at least two production steps (i) and at least one regeneration step (ii), and in which
in a production step (i), a starting gas mixture comprising the n-butenes is mixed with an oxygen-comprising gas and brought into contact with a heterogeneous, particulate multimetal oxide catalyst comprising molybdenum and at least one further metal as active composition in the fixed-bed reactor (R) and
in a regeneration step (ii), the heterogeneous, particulate multimetal oxide catalyst comprising molybdenum and at least one further metal as active composition is regenerated by passing an oxygen-comprising regeneration gas mixture over it and burning off the carbonaceous material deposited on the multimetal oxide catalyst, where
a regeneration step (ii) is carried out between two production steps (i) and where
a product gas stream which
comprises 1,3-butadiene and additionally unreacted n-butenes, oxygen, water and further secondary components, optionally hydrogen and optionally oxygenates is obtained in the production step (i) in the fixed-bed reactor (R) and is fed as such or after one or more intermediate steps as stream
to an absorption column (K) in which an absorption is carried out at a pressure in the range from 3.5 to 20 bar by means of a high-boiling absorption medium which becomes loaded with the C₄-hydrocarbons from the product gas stream or the stream and is taken off as loaded solvent stream from the bottom of the absorption column (K) to give an overhead stream comprising
oxygen, low-boiling hydrocarbons, optionally inert gases, in particular nitrogen, optionally carbon oxides and optionally water vapor, and is partly or completely recycled as recycle stream to the fixed-bed reactor (R),
wherein
at the end of each production step (i), the introduction of the oxygen-comprising gas (2) into the reactor (R) is throttled back or shut off and the production step (i) is continued until the oxygen concentration in the overhead stream has decreased to 5% by volume, based on the total volume of the overhead stream, whereupon
the introduction of the gas stream comprising the n-butenes
and also the introduction of the oxygen-comprising gas is shut off, if this has not already been done at the end of the production step (i),
at which point the production step (i) is complete and the regeneration step (ii) is started by the overhead stream from the absorption column (K) functioning as oxygen-comprising regeneration gas mixture or substream of the oxygen-comprising regeneration gas mixture.

9. The process as claimed in claim 8, wherein the regeneration step (ii) is carried out between two production steps (i) and where
a product gas stream which
comprises 1,3-butadiene and additionally unreacted n-butenes, oxygen, water and carbon monoxide, carbon dioxide, nitrogen, hydrocarbons having a boiling point of 95° C. or above at a pressure of one atmosphere, optionally hydrogen and optionally oxygenates is obtained in the production step (i) in the fixed-bed reactor (R) and is fed as such or after one or more intermediate steps as stream and
said overhead stream comprising
oxygen, hydrocarbons having a boiling point of less than 95° C. at a pressure of one atmosphere, residues of C₄-hydrocarbons, residues of high-boiling hydrocarbons having a boiling point of 95° C. or above at a pressure of one atmosphere, optionally nitrogen, optionally carbon oxides and optionally water vapor, and is partly or completely recycled as recycle stream to the fixed-bed reactor (R),
wherein
at the end of each production step (i), the introduction of the oxygen-comprising gas (2) into the reactor (R) is throttled back or shut off and the production step (i) is continued until the oxygen concentration in the overhead stream has decreased to 5% by volume, based on the total volume of the overhead stream, whereupon
the introduction of the gas stream comprising the n-butenes
and also the introduction of the oxygen-comprising gas is shut off, if this has not already been done at the end of the production step (i),
at which point the production step (i) is complete and the regeneration step (ii) is started by the overhead stream from the absorption column (K) functioning as oxygen-comprising regeneration gas mixture or substream of the oxygen-comprising regeneration gas mixture

10. The process according to claim 8, wherein the stream is fed as intermediate step to a quench (Q) in which the predominant part, of the high-boiling hydrocarbons is separated off at 20° C. and one atmosphere by direct contacting with a cooling medium and part of the water is separated off via the bottom stream to give a side stream which is fed as such or via a compressor (V) to the absorption column (K).

11. The process according to claim 8, wherein the stream is fed as intermediate step to a quench (Q) in at least 55% by volume, of the high-boiling hydrocarbons having a boiling point of 95° C. or above, is separated off at 20° C. and one atmosphere by direct contacting with a cooling medium and part of the water is separated off via the bottom stream to give a side stream which is fed as such or via a compressor (V) to the absorption column (K).

12. The process according to claim 8, wherein, as further intermediate step, the product gas stream is firstly fed to a compressor and brought therein to a pressure of from 3.5 to 20 bar absolute.

13. The process according to claim 8, wherein the process is carried out continuously.

14. The process according to claim 8, wherein the oxygen concentration of the regeneration gas mixture is increased during the course of the regeneration by introducing an oxygen-comprising stream.

15. The process according to claim 8, wherein the temperature is increased during the course of the regeneration.

16. The process according to claim 8, wherein the introduction of the oxygen-comprising gas into the reactor (R) is throttled back or shut off at the end of each production step (i) and the production step (i) is continued until the oxygen concentration in the overhead stream has decreased to 4.5% by volume, based on the total volume of the overhead stream.
